(19) United States
(12) Patent Application Publication
(10) Pub. No.: US 2022/0117782 A1
(43) Pub. Date: Apr. 21, 2022

(54) ONE-PIECE ADAPTOR DEVICE FOR OCULAR INSTILLATION OF OPHTHALMIC DROPS FROM A CONTAINER

(71) Applicant: JORGE BERNAL FIEGO, MATARO (ES)

(72) Inventor: JORGE BERNAL FIEGO, MATARO (ES)

(21) Appl. No.: 17/259,105

(22) PCT Filed: Jun. 27, 2019
(86) PCT No.: PCT/ES2019/070449

§ 371 Date: Jan. 8, 2021

(30) Foreign Application Priority Data
Jul. 12, 2018 (ES) U201831106

(51) Int. Cl. A61F 9/00; A61M 35/00; B05C 17/005

(52) U.S. Cl. CPC A61F 9/0026; B05C 17/005; A61M 35/00

(57) ABSTRACT

One-piece adaptor device for the administration of ophthalmic drops, which comprises an elongate alignment area which forms an alignment body of the adaptor device for receiving a single-dose container of ophthalmic fluid, the alignment area also comprising at least one means for holding said container, a support attachment joined as an extension to the alignment body which terminates in an ergonomic distal end, suitable for resting on the lower area of the eyelid, which adapter device is characterised in that it contains at least one protrusion on the alignment body for pressing the container and facilitating the ophthalmic fluid to come out by pressure.

The present application relates to an external adaptor device for use with a single-dose container for instilling an ophthalmic substance into the eye, in particular for instilling ophthalmic drops.

The use of single-dose devices for instilling ophthalmic substances is known. Ophthalmic drops are generally used to prevent or eliminate eye diseases such as dry eye, inflammation, glaucoma, allergies, the “red eye” effect owing to fatigue or tiredness or to prevent eye infections after an ophthalmic operation.

These ophthalmic substances are usually packed in a container, receptacle or vessel with a dispensing outlet for the application thereof. In addition, said containers have a “neck” or channel between said outlet and the container to regulate the amount delivered and to prevent mass delivery of said drops. Normally, to dispense the drops, the patient must turn the head back, position the container in the most correct direction possible to apply the drops and then apply force to the container to allow the drops to come out of the dispensing outlet of the container.

Many people have difficulty in self-administering the drops. This is especially true with regard to older people, those with physical disabilities or people who do not have much strength. In particular, one of the major drawbacks for such people consists in applying enough force to the container for the drops to come out while holding the single-dose device in one hand.

The walls of the single-dose devices are usually resilient and can be deformed by the user squeezing. However, the characteristics of said single-dose containers are not uniform in their design, and it is therefore common for many users to have difficulty in applying the pressure required to dispense the drop.

Moreover, another drawback consists in positioning the outlet in the correct direction for the eye drop to be applied correctly. Even in those who can achieve suitable positioning, there is a natural blink reflex which makes the process more difficult and may result in the drop being spilt and not applied in the eye.

All this has an economic cost owing to the waste of the ophthalmic substance and also uncertainty for the user, as he is often not sure if the ophthalmic substance was applied correctly.

In addition, in many cases there is the drawback that the patient often does not position the nozzle close enough to the eye for fear that said nozzle may touch the cornea, making it difficult to instil the drop correctly.

Embodiments exist in the prior art that are intended to make instillation into the eye easier and to overcome some of the drawbacks of the prior art. Spanish utility model document U9002445Y discloses a device, albeit multi-dose, for instilling ophthalmic substances into the eye that incorporates an adaptor extension for use as a support on the lower area of the eyelid to help keep the eye open during instillation.

The Austrian AT515704A1 and U.S. Pat. No. 5,133,702 patent applications disclose devices aimed at overcoming some of these drawbacks. However, they have the disadvantage of requiring a mechanism with various parts that move relative to each other; a larger number of parts in a mechanism represents a greater risk of said parts being detached, lost or broken, rendering the device inoperable if a replacement part is not obtained. In addition, these documents do not disclose a technical effect that avoids excessive turning of the patient's head during the process of applying the drops.

U.S. Pat. No. 5,429,621A discloses a device for overcoming some of these drawbacks. Said device has a surface on which the single-dose container is placed, an adaptor attachment which rests on the lower area of the eyelid and a mechanism for holding the container by means of two tabs which encircle the surface of the container. However, this system still has serious drawbacks. The means for making it easier for the patient to apply pressure consists of two parts on the sides or tabs, such that by placing the single-dose container on the base and closing the device for application thereof, it is possible to apply pressure accidentally to the walls of the container, causing the medicine to spill. Otherwise, the patient must place said container in position and then with the other hand try to close the tabs and apply pressure to the container. Moreover, said system still has the drawback that the patient will have to turn his head excessively to achieve correct positioning of the medicine, this being determined by the angle of the attachment relative to the container placement surface, which in said invention is zero.

The present invention attempts to overcome some of the above-mentioned problems of the prior art.

More particularly, the present invention discloses a one-piece adaptor device for instilling ophthalmic drops into the eye from a container, which comprises:
an elongate alignment area which forms an alignment body of the adaptor device for receiving a single-dose container of ophthalmic fluid, the alignment area also comprising at least one means for holding said container,
a support attachment joined as an extension to the alignment body which terminates in an ergonomic distal end, suitable for resting on the lower area of the eyelid,
at least one protrusion on the alignment body for pressing the container and facilitating the ophthalmic fluid to come out by pressure.

Preferably, the support attachment is tilted relative to the longitudinal axis of the alignment body. Preferably, said tilt has an angle relative to the longitudinal axis of the alignment body of between 1° and 90°. More preferably, said angle is between 10° and 35°.

Preferably, the protrusion on the alignment body has a transverse cross section in the form a triangular prism. In another preferred embodiment, said protrusion has a mound-shaped transverse cross section. In other embodiments, said protrusion has a rectangular cross section. Said protrusion may be any shape that helps apply pressure to the resilient walls of the container.

Preferably, the protrusion is arranged on the central portion on the alignment area. In other embodiments, the protrusion covers the entire transverse section of the alignment area.

The adaptor device at the ergonomic distal end, suitable for resting on the lower eyelid, is preferably shaped like a cylindrical wall. In another embodiment, said distal end is concave. In another preferred embodiment, the sides thereof widen. Said distal end may have any other ergonomic shape that helps improve contact with the lower eyelid.

Preferably, the means for holding the adaptor comprises a hollow aperture in the alignment area for receiving the rear extension of the single-dose container. Thus, by elastic resilience in the contact area of the extension of the container with the edges of the hollow aperture, the container will be maintained in the required position to provide said holding.

More preferably, the adaptor device will have an additional holding means which comprises L-shaped flanges arranged perpendicular to the alignment area from the side thereof with the shorter length. In an additional embodiment, said additional holding means comprises at least two walls arranged perpendicular to the upper face of the alignment area.

Preferably, at the join between the support attachment and the alignment body, the adaptor device comprises at least one protrusion on each of the sides that form a stop for the container, leaving a central space for the dispensing outlet of the container of ophthalmic fluid.

In an additional embodiment, the adaptor device will also have a gripping arm hinged to the alignment body. The gripping arm connects to the alignment body by means of a weakened area, leaving space for the dispensing nozzle or outlet. The gripping arm will also have at least one protrusion arranged perpendicular to the lower face of said gripping arm for facilitating the ophthalmic flow to come out by pressure.

For a better understanding, the accompanying drawings show as an explanatory but non-limiting example some of the embodiments of the present invention included in this document.

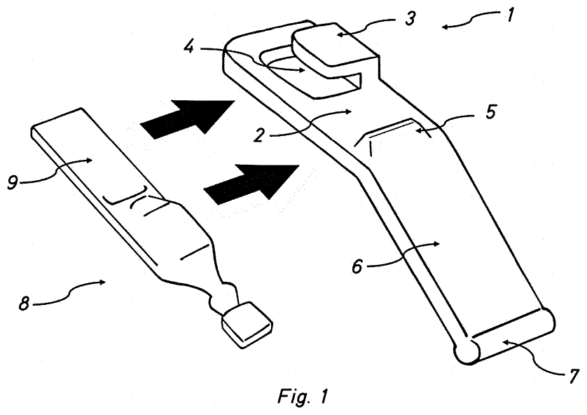

FIG. 1 is a perspective view of a single-dose container for eye drops in a first embodiment of the present invention.

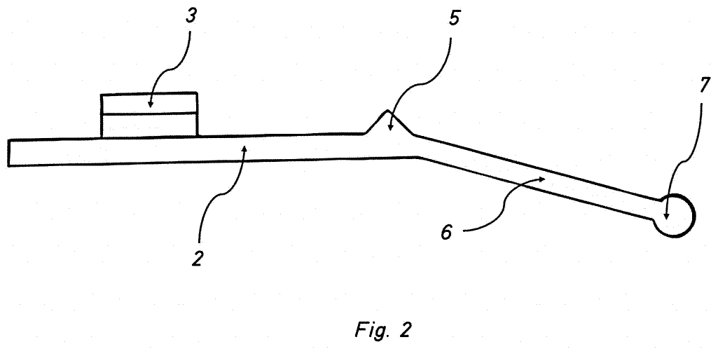

FIG. 2 is a side view of the embodiment shown in FIG. 1.

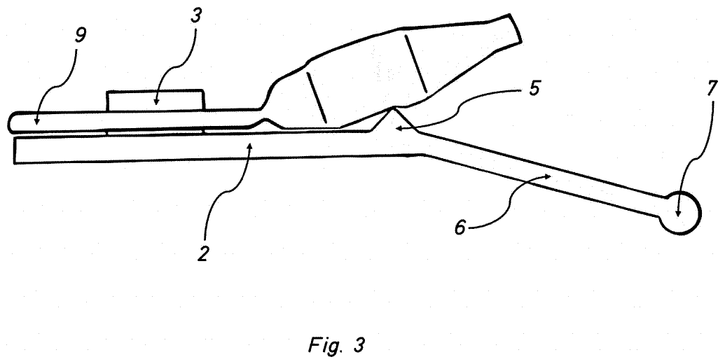

FIG. 3 is a side view of the embodiment according to FIGS. 1 and 2, with the single-dose container positioned on the alignment area.

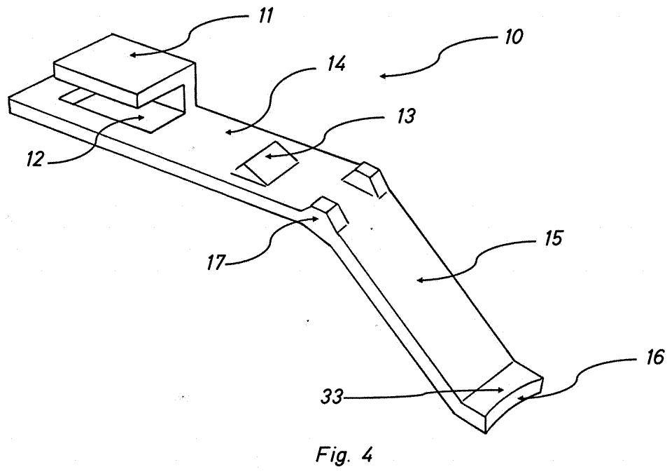

FIG. 4 is a perspective view of a second embodiment according to the present invention.

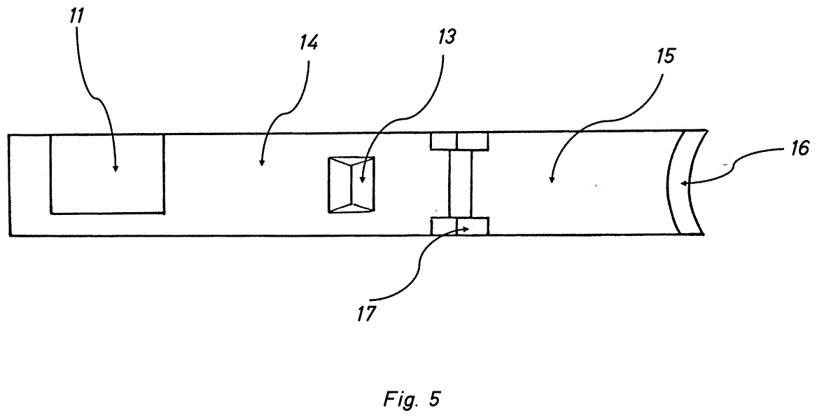

FIG. 5 is a plan view of the embodiment shown in FIG. 4.

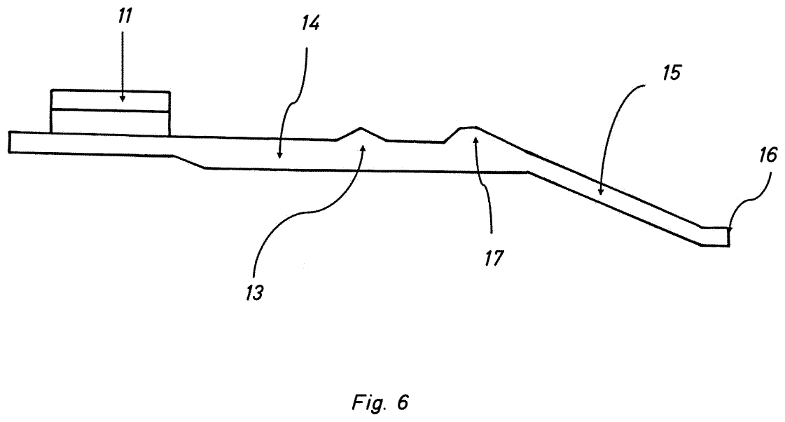

FIG. 6 is a side view of FIGS. 4 and 5.

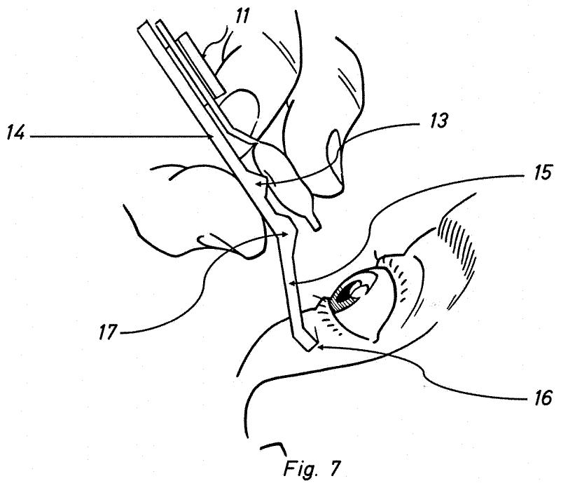

FIG. 7 is an example of use according to the embodiment shown in FIG. 4.

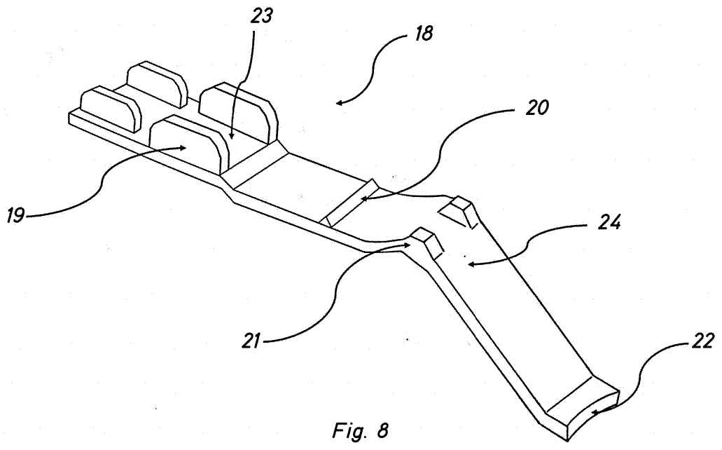

FIG. 8 is a perspective view of a third embodiment.

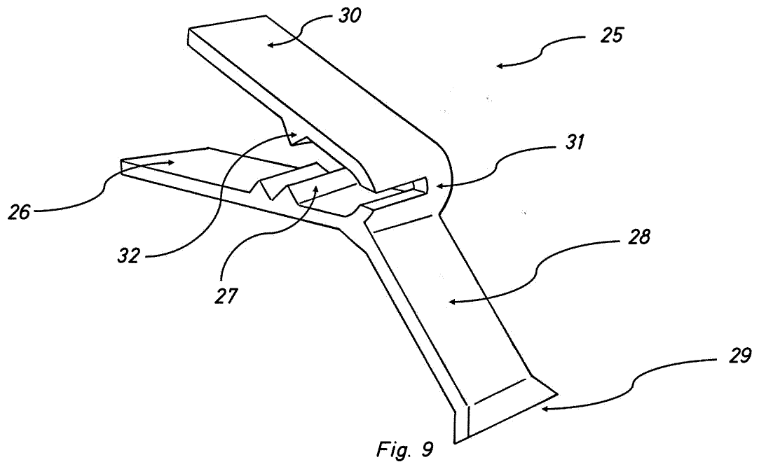

FIG. 9 is a perspective view of a fourth embodiment.

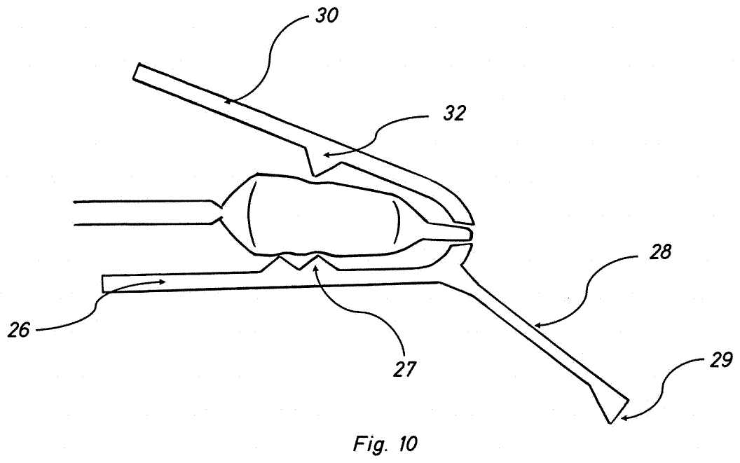

FIG. 10 is a side view of the embodiment shown in FIG. 9.

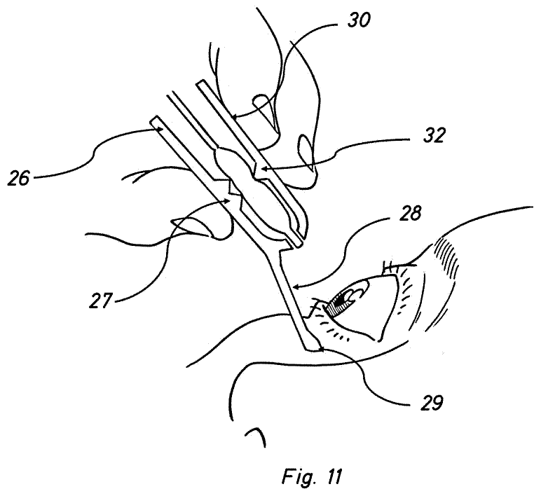

FIG. 11 is an example of use of the embodiment shown in FIGS. 9 and 10.

FIG. 1 is a perspective view of a first embodiment of the invention of an adaptor device -1- for a single-dose container -8- for instilling an ophthalmic substance into the eye. Said adaptor device is formed as a single body which comprises an alignment area -2- which extends longitudinally, forming an alignment body of the adaptor device for receiving a container -8- of ophthalmic fluid, the alignment area also comprising at least one means for holding said container -8-, which comprises a flange -3- arranged in an L shape, perpendicular to the alignment area from the side thereof with the shorter length. The extension -9- of the single-dose container is fitted into said flange. Preferably, the flange -3- is resilient and flexible in order better to adapt to the thickness of the extension -9- of different containers -8-. In addition, the present embodiment also has an additional holding means which consists of a hollow aperture -4- arranged in the alignment area -2-, said aperture -4- being suitable for receiving the rear extension -9- of the single-dose container and thus making said container easier to hold. The extension -9- of the container is inserted in said hollow aperture and by elastic resilience in the contact area at the edges of the hole will maintain the extension -9- in the required position to allow said holding. The present embodiment comprising both holding means simultaneously has significant advantages as, depending on the thickness of the extension of the single-dose container, which is not commercially universal, the patient may use the more suitable holding means.

The embodiment in FIG. 1 also comprises a support area which comprises a support attachment -6- joined as an extension to the alignment body; the support attachment -6- terminates in a rounded, approximately cylindrical, ergonomic distal end -7- suitable for resting on the lower eyelid. The support attachment is tilted relative to the longitudinal axis of the alignment body to facilitate the application thereof and to reduce uncomfortable turning of the head by the patient. This is particularly important for older patients or those with physical disabilities. In addition, said tilting has other technical effects such as optimal separation between the support on the lower eyelid and the nozzle of the container, which allows better centring of said nozzle, and also allows greater ease when adjusting the thumb during the application of the ophthalmic drops.

The adaptor device also comprises a protrusion -5- to facilitate the outflow by pressure; said protrusion has a transverse cross section in the form of a triangular prism.

FIG. 3 is a side view with the single-dose container positioned in the alignment area.

FIG. 4 shows a second embodiment of an adaptor device -10- for a single-dose container -8- for instilling an ophthalmic substance into the eye. The adaptor device -10- is formed of a single body which comprises an alignment area -14- which projects longitudinally forming an alignment body of the adaptor device intended to receive a single-dose container of ophthalmic fluid. The alignment area -14- comprises at least one means for holding said container which comprises a flange -11- arranged in an L shape, arranged perpendicular to the alignment area from the side thereof with the shorter length. Depending on the thickness of the extension -9- of the single-dose container, said container may be fitted to said flange. Preferably, the flange -11- is resilient and flexible, which allows better adaptation to the thickness of different containers -8-. In addition, the embodiment shown may also have an additional holding means which consist of an aperture -12- arranged in the alignment area -2- suitable for receiving the rear extension -9- of the single-dose container and thus facilitating the holding thereof.

The embodiment in FIG. 4 also comprises a support attachment -15- joined as an extension to the alignment body. The support attachment -15- is tilted relative to the longitudinal axis of the alignment body to facilitate the application thereof and reduce uncomfortable turning of the head by the patient. This is particularly important for older patients and those with physical disabilities. In addition, said tilt has other technical effects such as optimal separation between the support on the lower eyelid and the nozzle of the container, which allows better centring of said nozzle, and also allows greater ease when adjusting the thumb during the application of the ophthalmic drops. Said support attachment terminates in a bent portion -33-, which has a concave ergonomic distal end -16- suitable for resting on the lower eyelid.

The adaptor device comprises a protrusion -13-; said protrusion has a transverse cross section in the form of a triangular prism.

The embodiment in FIGS. 4 to 7, at the join between the support attachment and the alignment attachment, comprises a protrusion -17- on each of the sides, said protrusions -17- forming a stop for the container, leaving a central space for the dispensing outlet of the container of ophthalmic fluid.

FIG. 8 is a perspective view of a third embodiment of an adaptor device -18- for a single-dose container for instilling an ophthalmic substance into the eye. Said adaptor device -18- is formed of a single body which comprises an alignment area -23- which projects longitudinally forming an alignment body of the adaptor device for receiving a single-dose container of ophthalmic fluid. The alignment area comprises a means for holding said container -8-. The adaptor device comprises in the alignment area -23- a holding means which comprises at least two side walls -19- arranged perpendicular to the upper face of the support area.

The embodiment in FIG. 8 comprises a support area, which in turn comprises a support attachment -24- joined as an extension to the alignment body -23-. The support attachment -24- terminates in a concave ergonomic distal end -22-, suitable for resting on the lower eyelid. The support attachment -24- is tilted relative to the longitudinal axis of the alignment body -23- to make the application thereof easier and to reduce uncomfortable turning of the head by the patient. This is particularly important for older patients or those with physical disabilities. In addition, said tilting has other technical effects such as optimal separation between the support on the lower eyelid and the nozzle of the container, which allows better centring of said nozzle, and is also more convenient when adjusting the thumb during the application of the ophthalmic drops. In addition, the adaptor device comprises a protrusion -20- to facilitate the outflow of the ophthalmic fluid by pressure; said protrusion has a mound-shaped transverse cross section. Preferably, the protrusion -20- covers the entire transverse section of the alignment area. In addition, at the join between the support attachment and the adjustment attachment, at least one protrusion -21- is arranged on each of the sides which form a stop for the container, leaving a central space for the dispensing outlet of the container of ophthalmic fluid.

The embodiment in FIG. 9 is a perspective view of a fourth embodiment of an adaptor device -25- for a single-dose container -8- for instilling an ophthalmic substance into the eye, said adaptor device being formed of a single body and comprising an alignment area -26- which projects longitudinally forming an alignment body of the adaptor device for receiving a single-dose container of ophthalmic fluid. The alignment area comprises at least one means for holding said container.

The embodiment in FIG. 9 comprises a support area which in turn comprises a support attachment -28- arranged as an extension of the alignment body -26-. The support attachment is tilted relative to the longitudinal axis of the alignment body to facilitate the application thereof and to reduce uncomfortable turning of the head by the patient. This is particularly important for older patients or those with physical disabilities. In addition, said tilting has other technical effects such as optimal separation between the support on the lower eyelid and the nozzle of the container, which allows better centring of said nozzle, and also allows greater ease when adjusting the thumb during the application of the ophthalmic drops. The support attachment -28- terminates in an ergonomic distal end -29-, the sides of which widen, which is suitable for resting on the lower eyelid.

The adaptor device -25- has a gripping arm -30-. The gripping arm -30- is hinged to the alignment body -26- by means of a weakened area -31-. The dispensing outlet is arranged such that space is left for the nozzle or dispensing outlet of the single-dose container. The gripping arm -30- has at least one additional protrusion -32- arranged perpendicular to the lower face of the gripping body -30- which helps facilitate the outflow by pressure of the ophthalmic fluid.

The alignment body also comprises a protrusion -27- to facilitate the outflow by pressure; said protrusion has a transverse cross section in the form of a triangular prism. Said protrusion -27- preferably covers the entire transverse section of the alignment area. This embodiment consists of a one-piece adaptor device made by means of injection moulding of the same plastics material.

This embodiment allows greater ease in applying pressure by squeezing with a gripper, compared with doing so with the fingers, to the single-dose container, while at the same time having to hold the support surface.

Note that all the embodiments are made of a single part, thus avoiding mechanisms that could become damaged or detached, and parts that could be misplaced.

In addition, the embodiments of the present invention avoid the drawbacks of incorrect orientation of the nozzle, involuntary blinking by the patient and prevents the nozzle of the container from coming in contact with the patient's eye.

The invention is also particularly suitable for those patients who may have difficulty in applying the necessary pressure for dispensing, as it has a protrusion on the upper face of the alignment surface which, for the same force applied by the patient, will allow greater resilient deformation of the walls of the container to be produced. In addition, the strategic positioning of the protrusion on the upper face of the alignment area allows particularly practical application, while a one-piece adaptor device is also obtained.

Although the invention has been described and illustrated based on various representative embodiments, it should be understood that said embodiments are given as an example and in no way limit the present invention. Therefore any variations included directly or by equivalence in the content of the accompanying claims should be considered included within the scope of the present invention.

1. One-piece adaptor device for the administration of ophthalmic drops, which comprises:
an elongate alignment area which forms an alignment body of the adaptor device for receiving a single-dose container of ophthalmic fluid, the alignment area also comprising at least one means for holding said container,
a support attachment joined as an extension to the alignment body which terminates in an ergonomic distal end, suitable for resting on the lower area of the eyelid, and
at least one protrusion on the alignment body for pressing the container and facilitating the ophthalmic fluid to come out by pressure.

2. The one-piece adaptor device according to claim 1, wherein the support attachment is tilted relative to the longitudinal axis of the alignment body.

3. The one-piece adaptor device according to claim 1, wherein the angle of tilt is between 1° and 90°.

4. The one-piece adaptor device according to claim 1, wherein the angle of tilt is between 10° and 35°.

5. The one-piece adaptor device according to claim 1, wherein the protrusion has a transverse cross section in the form of a triangular prism.

6. The one-piece adaptor device according to claim 1, wherein the protrusion has a mound-shaped transverse cross section.

7. The one-piece adaptor device according to claim 1, wherein the protrusion covers the entire transverse section of the alignment area.

8. The one-piece adaptor device according to claim 1, wherein the protrusion is arranged only on the central portion of the alignment area.

9. The one-piece adaptor device according to claim 1, wherein the support attachment at the distal end is in the form of a cylindrical wall.

10. The one-piece adaptor device according to claim 1, wherein the support attachment is concave at the distal end thereof.

11. The one-piece adaptor device according to claim 1, wherein the sides of the support attachment widen at the distal end thereof.

12. The one-piece adaptor device according to claim 1, wherein the holding means comprises a hollow aperture in the alignment area for inserting a single-dose container.

13. The one-piece adaptor device according to claim 1, wherein the holding means comprises L-shaped flanges arranged perpendicular to the alignment area from the side thereof with the shorter length.

14. The one-piece adaptor device according to claim 1, wherein the holding means comprises at least two side walls arranged perpendicular to the upper face of the alignment area.

15. The one-piece adaptor device according to claim 1, wherein at the join between the support attachment and the alignment body, said adaptor device comprises at least one protrusion on each of the sides that form a stop for the container, leaving a central space for the dispensing outlet of the container of ophthalmic fluid.

16. The one-piece adaptor device according to claim 1 further comprising a gripping arm hinged to the alignment body.

17. The one-piece adaptor device according to claim 16, wherein said gripping arm connects to the alignment body by means of a weakened area, leaving space for the nozzle or dispensing outlet of the single-dose container.

18. The one-piece adaptor device according to claim 17, wherein the gripping arm has at least one additional protrusion arranged perpendicular to the lower face of the gripping attachment to facilitate the outflow of ophthalmic fluid.
